(19) United States
(12) Patent Application Publication
(10) Pub. No.: US 2016/0125766 A1
(43) Pub. Date: May 5, 2016

(54) PHOTOACOUSTIC BLOOD MODEL

(71) Applicant: CANON KABUSHIKI KAISHA, Ohta-ku, Tokyo (JP)

(72) Inventors: Ryo Ogawa, Kawasaki-shi (JP); Takahiro Suita, Kawasaki-shi (JP)

(73) Assignee: CANON KABUSHIKI KAISHA, Tokyo (JP)

(21) Appl. No.: 14/890,413

(22) PCT Filed: May 20, 2014
(86) PCT No.: PCT/JP2014/063765

§ 371 Date: Nov. 10, 2015

(30) Foreign Application Priority Data
May 23, 2013 (JP) 2013-108702
May 23, 2013 (JP) 2013-108703
Mar. 7, 2014 (JP) 2014-044541

(51) Int. Cl. G09B 23/28; A61B 5/00; G01N 29/30

(52) U.S. Cl. CPC G09B 23/286; G01N 29/30; A61B 5/0095; A61B 2560/0223

(57) ABSTRACT

The photoacoustic blood model contains two or more kinds of light absorbing compounds in a blood model base material, in which the absorption coefficient ratios μ[λ₂]/μ[λ₁] at arbitrary two wavelengths λ₁ and λ₂ (λ₁<λ₂) of 600 nm or more and 1100 nm or less of the compounds are different from each other and the parameter S calculated from Equation (1) is 0 or more and 100 or less, in which HbO₂[λ₁] indicates an absorption coefficient of oxyhemoglobin at the wavelength λ₁, HbO₂[λ₂] indicates an absorption coefficient of oxyhemoglobin at the wavelength λ₂, Hb[λ₁] indicates an absorption coefficient of deoxyhemoglobin at the wavelength λ₁, Hb[λ₂] indicates an absorption coefficient of deoxyhemoglobin at the wavelength λ₂, and P′ indicates a ratio (P_{λ2}/P_{λ1}) of a photoacoustic signal intensity P_{λ2} obtained by irradiation with light of the wavelength λ₂ to a photoacoustic signal intensity P_{λ1} obtained by irradiation with light of the wavelength λ₁.

### TECHNICAL FIELD

The present invention relates to a photoacoustic blood model and a phantom for a photoacoustic wave diagnosing apparatus which are used in accuracy control and calibration of a photoacoustic wave diagnosing apparatus.

### BACKGROUND ART

In recent years, a development of a photoacoustic wave diagnosing apparatus has been advanced as a diagnosing apparatus using light. The photoacoustic wave diagnosing apparatus is an apparatus used for medical diagnosis and is an apparatus which irradiates an examination portion of a living body with light, detects signals of acoustic waves (typically ultrasonic waves) resulting from thermal expansion of a measuring target, and then displays an image based on the detected signals. The diagnosing apparatus examines specific substances in the examination portion, e.g., glucose, hemoglobin, and the like contained in blood.

It is known that when a tumor such as a cancer grows in biological tissues, blood vessels around the tumor are newly formed and the oxygen is increasingly consumed by the tumor. As a method for evaluating the formation of new blood vessels and the increase in oxygen consumption, light absorption coefficients of oxyhemoglobin (HbO₂) and deoxyhemoglobin (Hb) can be utilized. For example, the photoacoustic wave diagnosing apparatus measures the concentration of HbO₂ and Hb in blood from the light absorption coefficients of HbO₂ and Hb at a plurality of wavelengths. Then, by creating a concentration distribution image of HbO₂ and Hb in the biological tissues, a region where the new blood vessels are formed can be identified. Moreover, by calculating the oxygen saturation degree based on the light absorption coefficient ratio of HbO₂ and Hb at at least two wavelengths, a region where the oxygen consumption increases, which is considered to be a region where the tumor is present, can be identified. For example, it is known that the oxygen saturation in the tumor region reaches about 70%. Moreover, it is suggested that there is a correlation between the malignancy of the tumor and the oxygen saturation. When identifying the malignancy of the tumor using the oxygen saturation, the accuracy which allows identification of a difference in the oxygen saturation is demanded.

Moreover, the photoacoustic wave diagnosing apparatus can also measure the blood flow velocity by measuring a delay of the arrival time of generated ultrasonic waves utilizing the photoacoustic Doppler effect produced by the movement of blood. Thus, the photoacoustic wave diagnosing apparatus can simultaneously measure the blood flow velocity and the oxygen saturation.

The accuracy control of such a diagnosing apparatus for medical use is indispensable in order to perform correct diagnosis, and a standard sample for use in accuracy control and calibration of the diagnosing apparatus, i.e., a phantom, has been used for the purpose. In the case of the photoacoustic wave diagnosing apparatus, accuracy control and calibration can be performed using a blood model which has light absorption characteristics simulating a tumor present in the tissues and transmits acoustic waves based on the light absorption similarly as in the biological tissues. Heretofore, in a photoacoustic phantom, the light absorption is simulated using India ink or the like. For example, NPL 1 discloses a method of dispersing carbon nanotubes in a gel of alginic acid salt, and controlling the absorption coefficient. Moreover, for an optical diagnosing apparatus (for example, pulse oximeter) which performs diagnosis of an examination portion based on the amount of light absorption, a method of managing the apparatus using an optical phantom in which oxygen saturation is controlled is disclosed, for example, as described in in PTL 1.

In the case of a photoacoustic wave diagnosing apparatus which measures oxygen saturation and blood flow velocity, accuracy control and calibration of the apparatus can be performed by the use of a blood-simulated fluid which has light absorption characteristics simulating those of blood vessels present in a living body and can move as fluid similarly to blood. As such a photoacoustic phantom used to measure the blood flow velocity, a phantom which simulates the light absorption using India ink or the like is mentioned. For example, in NPL 1, a phantom which simulates the blood flow velocity obtained by dispersing amorphous carbon powder in water for absorption is used.

### CITATION LIST

### Patent Literature

PTL 1 Japanese Patent No. 2683848

### Non Patent Literature

NPL 1 Journal of Biomedical Optics 16(5), 051304

NPL 2 Physical Review Letters Vol. 99, 184501 (2007)

### Technical Problem

However, according to the method of NPL 1, a compound having light absorbance is carbon nanotubes and the absorption at each measurement wavelength is fixed and the absorption coefficient ratio at different wavelengths cannot simulate the absorption coefficient ratio of HbO₂ and Hb. Moreover, in PTL 1, since a paint having fluorescent characteristics is used, a luminous phenomenon according to the light absorption is involved. Therefore, when used as a photoacoustic phantom, PTL 1 has a problem of noise generated by fluorescence luminescence.

According to the method of NPL 2, a compound having light absorbance is carbon, and the absorption at each measurement wavelength is fixed and the absorption coefficient ratio at different wavelengths cannot simulate the absorption coefficient ratio of HbO₂ and Hb. Moreover, in PTL 1, although the paint having fluorescent characteristics is used, only a dispersing element to a solid is disclosed, and thus it is difficult to use the dispersing element as a photoacoustic fluid phantom for use in management and calibration of a photoacoustic wave diagnosing apparatus which measures the oxygen saturation and the blood flow velocity.

The present invention provides a photoacoustic blood model and a phantom for a photoacoustic wave diagnosing apparatus which can simulate the oxygen saturation of a human body and is suitable for accuracy control and calibration of a photoacoustic wave diagnosing apparatus.

### SUMMARY OF INVENTION

### Solution to Problem

The present inventors have conducted extensive research, and as a result found that a value of a parameter S calculated from the following expression (1) can be controlled in the range of 0 or more and 100 or less by compounding two or more kinds of specific light absorbing compounds. Thus, the present inventors have accomplished the present invention.

More specifically, a photoacoustic blood model of the present invention contains two or more kinds of light absorbing compounds in a blood model base material, in which the absorption coefficient ratios μ[λ₂]/μ[λ₁] at arbitrary two wavelengths λ₁ and λ₂ (λ₁<λ₂) of 600 nm or more and 1100 nm or less of the light absorbing compounds are different from each other and the parameter S calculated from the following equation (1) is 0 or more and 100 or less.

$$\begin{matrix}[ Math . 1 ] & \\ S = \frac{{P}^{′} \cdot Hb [ {λ}_{1} ] - Hb [ {λ}_{2} ]}{\begin{matrix}( {HbO}_{2} [ {λ}_{2} ] - Hb [ {λ}_{2} ] ) - \\ {P}^{′} \cdot ( {HbO}_{2} [ {λ}_{1} ] - Hb [ {λ}_{1} ] )\end{matrix}} \cdot 100 & Expression ( 1 )\end{matrix}$$

HbO₂[λ₁]: Absorption coefficient of oxyhemoglobin at the wavelength λ₁
HbO₂[λ₂]: Absorption coefficient of oxyhemoglobin at the wavelength λ₂
Hb[λ₁]: Absorption coefficient of deoxyhemoglobin at the wavelength λ₁
Hb[λ₂]: Absorption coefficient of deoxyhemoglobin at the wavelength λ₂
P′: Ratio (P_{λ2}/P_{λ1}) of the photoacoustic signal intensity Paz obtained by irradiation with light of the wavelength λ₂ to the photoacoustic signal intensity P_{λ}1 obtained by irradiation with light of the wavelength λ₁

Moreover, the phantom for a photoacoustic wave diagnosing apparatus of the present invention has the above-described photoacoustic blood model and a phantom base material.

Further features of the present invention will become apparent from the following description of exemplary embodiments with reference to the attached drawings.

### Advantageous Effects of Invention

In the photoacoustic blood model of the present invention, when measured with a photoacoustic wave diagnosing apparatus, the parameter S value can be controlled in the range of 0 or more and 100 or less. The parameter S is a value equivalent to the oxygen saturation of a human body. The photoacoustic blood model and the phantom for a photoacoustic wave diagnosing apparatus of the present invention can simulate the oxygen saturation of a human body in photoacoustic wave diagnosis and is suitable for use in accuracy control and calibration of a photoacoustic wave diagnosing apparatus.

### BRIEF DESCRIPTION OF DRAWINGS

FIG. 1 is a view illustrating a configuration example of a phantom for a photoacoustic wave diagnosing apparatus employing a photoacoustic blood model of the present invention.

FIG. 2 is a view illustrating a configuration example of a phantom for a photoacoustic wave diagnosing apparatus employing a photoacoustic fluid blood model of the present invention.

FIG. 3 is an outline view of a photoacoustic signal intensity measuring device.

FIG. 4 is a view illustrating the waveform of a typical photoacoustic signal.

### DESCRIPTION OF EMBODIMENT

Hereinafter, the present invention is described. The embodiment to be disclosed is one example of the present invention and the present invention is not limited thereto. In the present invention, “living bodies”, such as a “human body”, include not only a living body but a cut-out pathology site and the like.

### Photoacoustic Blood Model

The photoacoustic blood model of the present invention (sometimes referred to as a “blood model”) contains two or more kinds of light absorbing compounds in a blood model base material.

### Light Absorbing Compound

In the light absorbing compounds, the absorption coefficient ratios μ[λ₂]/μ[λ₁] at arbitrary two wavelengths λ₁ and λ₂ (λ₁<λ₂) of 600 nm or more and 1100 nm or less are different from each other.

In the photoacoustic blood model of the present invention, a parameter S calculated from the following expression (1) can be controlled in the range of 0 or more and 100 or less by compounding two or more kinds of light absorbing compounds different in μ[λ₂]/μ[λ₁].

$$\begin{matrix}[ Math . 2 ] & \\ S = \frac{{P}^{′} \cdot Hb [ {λ}_{1} ] - Hb [ {λ}_{2} ]}{\begin{matrix}( {HbO}_{2} [ {λ}_{2} ] - Hb [ {λ}_{2} ] ) - \\ {P}^{′} \cdot ( {HbO}_{2} [ {λ}_{1} ] - Hb [ {λ}_{1} ] )\end{matrix}} \cdot 100 & Expression ( 1 )\end{matrix}$$

HbO₂[λ₁]: Absorption coefficient of oxyhemoglobin at the wavelength λ₁
HbO₂[λ₂]: Absorption coefficient of oxyhemoglobin at the wavelength λ₂
Hb[λ₁]: Absorption coefficient of deoxyhemoglobin at the wavelength λ₁
Hb[λ₂]: Absorption coefficient of deoxyhemoglobin at the wavelength λ₂
P′: Ratio (P_{λ2}/P_{λ1}) of the photoacoustic signal intensity P_{λ2} obtained by irradiation with light of the wavelength λ₂ to the photoacoustic signal intensity P_{λ1} obtained by irradiation with light of the wavelength λ₁

The value of the absorption coefficient of oxyhemoglobin and deoxyhemoglobin at each wavelength can be obtained by the following method. More specifically, a solution in which oxyhemoglobin or deoxyhemoglobin is 100% can be prepared by adjusting the oxygen partial pressure in an aqueous solution containing hemoglobin of a certain fixed concentration. The absorption coefficient at each wavelength can be obtained by measuring the solution with a spectrum photometer.

The wavelength range of 600 nm or more and 1100 nm or less is a range referred to as a so-called “Biological window”. The light having the wavelength in this range efficiently penetrates a human body and is suitable for use in a photoacoustic wave diagnosing apparatus.

The parameter S is a value equivalent to the oxygen saturation of a human body. The photoacoustic blood model of the present invention can simulate the oxygen saturation of a human body in photoacoustic wave diagnosis and is suitable for accuracy control and calibration of a photoacoustic wave diagnosing apparatus.

Hereinafter, the respect that the photoacoustic blood model of the present invention can control the parameter S calculated from Expression (1) in the range of 0 or more and 100 or less is described in detail.

First, the absorption coefficient at the wavelengths λ₂ and λ₁ of the blood model can be arbitrarily adjusted by compounding two or more kinds of light absorbing compounds different in μ[λ₂]/μ[λ₁] with an arbitrary ratio, and a parameter S′ calculated from the following expression (1′) can be adjusted.

$$\begin{matrix}[ Math . 3 ] & \\ {S}^{′} = \frac{( {μ}_{a} [ {λ}_{2} ] / {μ}_{a} [ {λ}_{1} ] ) \cdot Hb [ {λ}_{1} ] - Hb [ {λ}_{2} ]}{\begin{matrix}( {HbO}_{2} [ {λ}_{2} ] - Hb [ {λ}_{2} ] ) - \\ ( {μ}_{a} [ {λ}_{2} ] / {μ}_{a} [ {λ}_{1} ] ) \cdot ( {HbO}_{2} [ {λ}_{1} ] - Hb [ {λ}_{1} ] )\end{matrix}} \cdot 100 & {( 1 )}^{′}\end{matrix}$$

HbO₂[λ₁]: Absorption coefficient of oxyhemoglobin at the wavelength λ₁
HbO₂[λ₂]: Absorption coefficient of oxyhemoglobin at the wavelength λ₂
Hb[λ₁]: Absorption coefficient of deoxyhemoglobin at the wavelength λ₁
Hb[λ₂]: Absorption coefficient of deoxyhemoglobin at the wavelength λ₂
μₐ[λ₁]: Absorption coefficient of the blood model at the wavelength λ₁
μₐ[λ₂]: Absorption coefficient of the photoacoustic blood model at the wavelength λ₂

More specifically, the absorption coefficients of the light absorbing compounds A, B, C, . . . at the wavelengths λ₁ and λ₂ are defined as μ_{A}[λ₁], μ_{B}[λ₁], μ_{C}[λ₁] . . . and μ_{A} [λ₂], μ_{B}[λ₂], μ_{C}[λ₂] . . . , respectively. The content concentrations of the light absorbing compounds A, B, C . . . in the blood models are defined as C_{A}, C_{B}, C_{C}, . . . , respectively. Then, the following relationship is established between the absorption coefficient and the content concentration of the light absorbing compound and the absorption coefficients μₐ[λ₁] and μₐ[λ₂] of the blood model:

μₐ[λ₁]=C_{A}·μ_{A}[λ₁]+C_{B}μ_{B}[λ₁]+C_{C}μ_{C}[λ₁]+ . . . ,

μₐ[λ₂]=C_{A}·μ_{A}[λ₂]+C_{B}·μ_{B}[λ₂]+C_{C}·μ_{C}[λ₂]+ . . .

When the absorption coefficient ratios μ_{A}[λ₂]/μ_{A}[λ₁], μ_{B}[λ₂]/μ_{B}[λ₁], μ_{c}[λ₂]/μ_{c}[λ₁] . . . of the light absorbing compounds A, B, C, . . . are fixed, the absorption coefficient ratio μₐ[λ₂]/μₐ[λ₁] of the blood models is fixed, and therefore, the parameter S′ cannot be controlled. Therefore, the absorption coefficient ratios at the wavelengths λ₁ and λ₂ of the light absorbing compounds A, B, C, . . . are required to be different from each other.

Therefore, by controlling the content concentration of the light absorbing compounds A, B, C, . . . in which the absorption coefficient ratios at the wavelengths λ₁ and λ₂ are different from each other in the blood models, the absorption coefficient ratio μₐ[λ₂]/μₐ[λ₁] of the blood models can be controlled. As a result, the parameter S′ can be adjusted.

Herein, when the blood models are irradiated with light of the wavelengths λ₁ and λ₂, acoustic waves (generally ultrasonic waves) are generated because the blood models thermally expand according to the absorption coefficient. The relationship of Px=Γ·μx·Fx is established between the intensity Px of the acoustic waves generated when irradiated with laser light of a certain wavelength x, the intensity Fx of the laser light in that case, and the absorption coefficient μx. Γ is referred to as a Gruneisen coefficient and is a constant peculiar to materials. Therefore, when the intensity Fx of the laser light is fixed, a proportionality relationship is established between the intensity Px of the acoustics wave and the absorption coefficient μx. Therefore, the ratio P(P_{λ2}/P_{λ1}) of the photoacoustic signal intensities P_{λ1} and P_{λ2} is the same value as μₐ[λ₂]/μₐ[λ₁], and thus S′=S is established.

Therefore, by the use of two or more kinds of light absorbing compounds in which the absorption coefficient ratios at the wavelengths λ₁ and λ₂ are different from each other, the photoacoustic blood model of the present invention can control the parameter S in Expression (1) in the range of 0 or more and 100 or less.

The light absorbing compound is a substance having light absorbance in a wavelength region of 600 nm or more and 1100 nm or less. The light absorbing compound is desirably a pigment from the viewpoint of weather resistance. However, in addition thereto, known colorants, such as dyes and pigments, can be used.

The light absorption characteristics of the light absorbing compound can be selected as appropriate based on the ratio of the absorption coefficients of oxyhemoglobin (HbO₂) and deoxyhemoglobin (Hb) at the wavelengths λ₁ and λ₂. More specifically, when the intensity Fx of the laser light is fixed as described above, S′=S is established. Therefore, the absorption coefficient ratio μₐ[λ₂]/μₐ[λ₁] of the blood models is defined as in the following expression based on Expression (1′):

μₐ[λ₂]/μₐ[λ₁]=((S/100)·HbO₂[λ₂]+(1−S/100)·Hb[λ₂])/((S/100)·HbO₂[λ₁]+(1−S/100)·Hb[λ₁]).

In this case, when the parameter S is 0 or more and 100 or less, μₐ[λ₂]/μₐ[λ₁] takes a value between Hb[λ₂]/Hb[λ₁] and HbO₂[λ₂]/HbO₂[λ₁]. Therefore, the ratio μₐ[λ₂]/μₐ[λ₁] of the absorption coefficient of the blood model to be controlled is determined based on the value of the absorption coefficient of hemoglobin at a wavelength to be used. The light absorbing compound of the present invention can be selected as appropriate based on the absorption coefficient ratio of the hemoglobin.

According to the intended use of a photoacoustic wave diagnosing apparatus, the ranges of the parameter S required for the blood model varies. Therefore, when the lower limit of the parameter S is defined as Sₘᵢₙ and the upper limit of the parameter S is defined as Sₘₐₓ according to the intended use of a diagnosing apparatus, it is suitable to contain at least one light absorbing compound in which the absorption coefficient ratio μ[λ₂]/μ[λ₁] satisfies either one of the following expression (2) or expression (3). Furthermore, it is more suitable to contain at least one light absorbing compound in which the absorption coefficient ratio μ[λ₂]/μ[λ₁] satisfies the other one of the following expression (2) or expression (3).

$$\begin{matrix}[ Math . 4 ] & \\ {S}_{min} \geq \frac{( μ [ {λ}_{2} ] / μ [ {λ}_{1} ] ) \cdot Hb [ {λ}_{1} ] - Hb [ {λ}_{2} ]}{\begin{matrix}( {HbO}_{2} [ {λ}_{2} ] - Hb [ {λ}_{2} ] ) - \\ ( μ [ {λ}_{2} ] / μ [ {λ}_{1} ] ) \cdot ( {HbO}_{2} [ {λ}_{1} ] - Hb [ {λ}_{1} ] )\end{matrix}} \cdot 100 & Expression ( 2 ) \\ {S}_{max} \leq \frac{( μ [ {λ}_{2} ] / μ [ {λ}_{1} ] ) \cdot Hb [ {λ}_{1} ] - Hb [ {λ}_{2} ]}{\begin{matrix}( {HbO}_{2} [ {λ}_{2} ] - Hb [ {λ}_{2} ] ) - \\ ( μ [ {λ}_{2} ] / μ [ {λ}_{1} ] ) \cdot ( {HbO}_{2} [ {λ}_{1} ] - Hb [ {λ}_{1} ] )\end{matrix}} \cdot 100 & Expression ( 3 )\end{matrix}$$

Sₘᵢₙ: Lower limit of parameter S
Sₘₐₓ: Upper limit of parameter S
HbO₂[λ₁]: Absorption coefficient of oxyhemoglobin at the wavelength λ₁
HbO₂[λ₂]: Absorption coefficient of oxyhemoglobin at the wavelength λ₂
Hb[λ₁]: Absorption coefficient of deoxyhemoglobin at the wavelength λ₁
Hb[λ₂]: Absorption coefficient of deoxyhemoglobin at the wavelength λ₂
μ[λ₁]: Absorption coefficient of the light absorbing compound at the wavelength μ₁
μ[λ₂]: Absorption coefficient of the light absorbing compound at the wavelength λ₂

Hereinafter, the light absorbing compound is specifically described with reference to a case where light of λ₁=756 nm and light of λ₂=797 nm are used but the present invention is not limited thereto.

The absorption coefficient Hb[λ₁] at 756 nm of deoxyhemoglobin is 1560.48×10⁻⁶ mm⁻¹, the absorption coefficient Hb[λ₂] at 797 nm is 792.66×10⁻⁶ mm⁻¹, the absorption coefficient HbO₂[λ₁] at 756 nm of oxyhemoglobin is 562.00×10⁻⁶ mm⁻¹, and the absorption coefficient HbO₂[λ₂] at 797 nm is 768.80×10⁻⁶ mm⁻¹. Therefore, in order to set the parameter S of the blood model in the range of 0 or more and 100 or less, the absorption coefficient ratio μₐ[λ₂]/μₐ[λ₁] of the blood model is required to be in the range of 0.51 or more and 1.37 or less. Therefore, μ[λ₂]/μ[λ₁] of one light absorbing compound is suitably 0.51 or less and μ[λ₂]/μ[λ1] of the other light absorbing compound is suitably 1.37 or more. A substance whose absorption coefficient ratio is not included in this range can be used for adjusting the absorption coefficient.

As a pigment having such absorption characteristics, the following known pigments can be mentioned. Blue pigments include phthalocyanine pigments of phthalocyanine compounds substituted or not substituted by metal and the like and anthraquinone pigments. Red pigments include a monoazo pigment, a disazo pigment, an azo lake pigment, a benzimidazolone pigment, a perylene pigment, a diketopyrrolopyrrole pigment, a condensed azo pigment, an anthraquinone pigment, a quinacridone pigment, and the like. Green pigments include a phthalocyanine pigment, an anthraquinone pigment, and a perylene pigment similarly as in the blue pigments. Yellow pigments include a monoazo pigment, a disazo pigment, a condensed azo pigment, a benzimidazolone pigment, an isoindolinone pigment, an anthraquinone pigment, and the like. Furthermore, black pigments include Pigment Black 7, carbon black, and the like. In addition thereto, purple, orange, and brown pigments can also be utilized.

Among the above, a phthalocyanine compound, particularly copper phthalocyanine which is a copper-substituted phthalocyanine compound, can be suitably used because μ[λ₂]/μ[λ₁] is 0.51 or less and is close to 0, and therefore the controllability is good. The content of the phthalocyanine compound is not particularly limited and is suitably 0.0000001% by weight or more and 0.1% by weight or less. Carbon black has μ[λ₂]/[λ₁] close to 1 and can be suitably used for adjusting the absorption coefficient at each wavelength. Therefore, it is suitable to use copper phthalocyanine and carbon black as the light absorbing compound.

The phthalocyanine compound can move the maximum absorption wavelength according to a metal type to be substituted and the aggregation state of the phthalocyanine compound and is suitable for controlling the absorption coefficient in the range of 600 to 1100 nm. Therefore, it is suitable to use a plurality of kinds of phthalocyanine compounds as the light absorbing compound and it is more suitable to use a phthalocyanine compound whose maximum absorption wavelength is λ₁ or less and a phthalocyanine compound whose maximum absorption wavelength is λ₂ or more. In this case, the content of each phthalocyanine compound is not particularly limited and is suitably 0.0000001% by weight or more and 0.1% by weight or less. As the phthalocyanine compound, copper phthalocyanine, a phthalocyanine vanadium complex, titanylphthalocyanine, and the like can be suitably used. It is more suitable to use copper phthalocyanine and a phthalocyanine vanadium complex in combination.

The light absorbing compound can be compounded by adding a mixture of a dispersing agent having affinity with the light absorbing compound, for example, a dispersing agent containing a polyol component, and the light absorbing compound to the blood model base material. The dispersing agent having affinity with the light absorbing compound suitably has an anion group for improving the dispersibility of the light absorbing compound. As the anion group, a sulfonyl group and a carboxyl group are more suitably used. As the amount of the anion group, the anion group is suitably contained in such an amount that the anion group can disperse the light absorbing compound. Since the amount of the anion group affects the affinity to the blood model base material, the amount is selected as appropriate according to the property of the blood model base material. The polyol includes polyether polyol, polyester polyol, and the like, for example, and is selected as appropriate considering the affinity with the blood model base material. Blood model base material

When the photoacoustic blood models are irradiated with light having a certain wavelength λ, the photoacoustic blood models thermally expand according to the absorption coefficient, so that acoustic waves (generally ultrasonic waves) are generated. Between the intensity P of the acoustic wave to be obtained, the intensity F of the laser light in that case, and the absorption coefficient μ, the relationship of P=Γ·μ·F is established. Γ is referred to as a Gruneisen coefficient and is a constant peculiar to materials.

In the blood model base material of the present invention, the Gruneisen coefficient Γ is important and is suitably similar to that of the living body. Γ is suitably 0.1 or more and 2.0 or less. Since Γ of biological soft tissues is around 1.0, Γ is more suitably 0.5 or more and 1.5 or less.

In the present invention, the absorption coefficient of the blood model is required to be adjusted by compounding a light absorbing compound in the blood model base material. Therefore, as the blood model base material simple substance, in the used wavelength band of the photoacoustic wave diagnosing apparatus, the light absorption is suitably small and transparent.

Moreover, the Gruneisen coefficient Γ has a relationship of Γ=β·v²/Cp (β: Coefficient of thermal volume expansion, v: Acoustic velocity, Cp: Specific heat at constant pressure).

The coefficient of thermal volume expansion β of the blood model base material can be generally considered to be β=3·α (α: Coefficient of linear thermal expansion). The coefficient of linear thermal expansion α of a general engineering plastic is 100 ppm/K or less. However, since the Gruneisen coefficient Γ becomes small in this case, the acoustic wave generated by light becomes weak, and thus it is not suitable as the blood model base material. Therefore, the coefficient of linear thermal expansion α of the blood model base material is suitably 100 ppm/K or more and 1000 ppm/K or less and more suitably 200 ppm/K or more and 500 ppm/K or less from the viewpoint of the shape maintenance properties of the blood model.

Since the acoustic velocity of biological tissues is in the range of about 1000 m/s to 1700 m/s, the acoustic velocity v of the blood model base material is suitably 800 m/s or more and 2000 m/s or less and more suitably 1300 m/s or more and 1700 m/s or less particularly considering the similarity of the acoustic propagation to soft tissues.

The specific heat at constant pressure Cp of the blood model base material is suitably in the range where the Gruneisen coefficient Γ does not deviate from that of a living body according to the coefficient of linear thermal expansion α because the specific heat of the biological soft tissues is 3.5 J/gK, which is greatly different from that of general materials.

As a material having such a physical property value, polymer materials, such as urethane resin, silicone resin, epoxy resin, acrylic resin, polyvinyl chloride, epoxy resin, polyethylene, nylon, natural rubber, polystyrene, and polybutadiene, can be mentioned but the material is not limited thereto. Among the above, particularly, a polyurethane gel which is one kind of a thermosetting urethane resin has an acoustic velocity v of about 1400 m/s, a coefficient of linear thermal expansion α of about 300 ppm/K, and a Gruneisen coefficient Γ of about 1.0 and is suitable as the blood model base material of the present invention.

A curable urethane gel is typically obtained by reacting polyol and polyisocyanate but the invention is not limited thereto.

The polyol is not particularly limited insofar as it has two or more hydroxyl groups in the molecule and an arbitrary suitable polyol can be adopted. For example, polyester polyol, polyether polyol, polyacryl polyol, and the like are mentioned. These substances can be used singly or in combination of two or more kinds thereof.

The polyester polyol is typically obtained by reacting a polybasic acid component and a polyol component.

The polybasic acid component includes, for example, aromatic dicarboxylic acid, such as orthophthalic acid, isophthalic acid, terephthalic acid, 1,4-naphthalene dicarboxylic acid, 2,5-naphthalene dicarboxylic acid, 2,6-naphthalene dicarboxylic acid, biphenyl dicarboxylic acid, and tetrahydrophthalic acid; aliphatic dicarboxylic acid, such as oxalic acid, succinic acid, malonic acid, glutaric acid, adipic acid, pimelic acid, suberic acid, azelaic acid, sebacic acid, decane dicarboxylic acid, dodecane dicarboxylic acid, octadecane dicarboxylic acid, tartaric acid, alkyl succinic acid, linoleic acid, maleic acid, fumaric acid, mesaconic acid, citraconic acid, and itaconic acid; alicyclic dicarboxylic acid, such as hexahydrophthalic acid, tetrahydrophthalic acid, 1,3-cyclohexanedicarboxylic acid, and 1,4-cyclohexanedicarboxylic acid; or reactive derivatives, such as acid anhydrides, alkyl esters, and acid halides thereof, and the like. These substances can be used singly or in combination of two or more kinds thereof.

The polyol component includes ethylene glycol, 1,2-propane diol, 1,3-propane diol, 1,3-butane diol, 1,4-butane diol, neopentyl glycol, pentane diol, 1,6-hexane diol, 1,8-octane diol, 1,10-decane diol, 1-methyl-1,3-butylene glycol, 2-methyl-1,3-butylene glycol, 1-methyl-1,4-pentylene glycol, 2-methyl-1,4-pentylene glycol, 1,2-dimethyl-neopentyl glycol, 2,3-dimethyl-neopentyl glycol, 1-methyl-1,5-pentylene glycol, 2-methyl-1,5-pentylene glycol, 3-methyl-1,5-pentylene glycol, 1,2-dimethyl butylene glycol, 1,3-dimethyl butylene glycol, 2,3-dimethyl butylene glycol, 1,4-dimethyl butylene glycol, diethylene glycol, triethylene glycol, polyethylene glycol, dipropylene glycol, polypropylene glycol, 1,4-cyclohexanedimethanol, 1,4-cyclohexanediol, bisphenol A, bisphenol F, hydrogenated bisphenol A, hydrogenated bisphenol F, and the like. These substances can be used singly or in combination of two or more kinds thereof.

The polyether polyol is typically obtained by adding alkylene oxide to polyhydric alcohol by performing ring opening polymerization. The polyhydric alcohol includes, for example, ethylene glycol, diethylene glycol, propylene glycol, dipropylene glycol, glycerin, trimethylol propane, and the like. The alkylene oxide includes, for example, ethylene oxide, propylene oxide, butylene oxide, styrene oxide, tetrahydrofuran, and the like. These substances can be used singly or in combination of two or more kinds thereof.

The polyacrylpolyol is typically obtained by copolymerizing (meth)acrylate and a monomer having a hydroxyl group. The (meth)acrylate includes, for example, methyl(meth)acrylate, butyl(meth)acrylate, 2-ethylhexyl(meth)acrylate, cyclohexyl(meth)acrylate, and the like. The monomer having a hydroxyl group includes, for example, hydroxy alkyl ester of (meth)acrylic acid, such as 2-hydroxyethyl(meth)acrylate, 2-hydroxypropyl(meth)acrylate, 3-hydroxypropyl(meth)acrylate, 2-hydroxybutyl(meth)acrylate, 4-hydroxybutyl(meth)acrylate, and 2-hydroxypentyl(meth)acrylate; monoester(meth)acrylate of polyhydric alcohol, such as glycerin and trimethylol propane; N-methylol(meth)acryl amide; and the like. These substances can be used singly or in combination of two or more kinds thereof.

For the polyacrylpolyol, other monomers may be copolymerized in addition to the monomer components mentioned above. As other monomers, arbitrary suitable monomers can be adopted insofar as the monomers can be copolymerized. Specific examples of the monomers include unsaturated monocarboxylic acid, such as (meth)acrylic acid; unsaturated dicarboxylic acid, such as maleic acid and an anhydride thereof or mono or diesters thereof; unsaturated nitriles, such as (meth)acrylonitrile; unsaturated amides, such as (meth)acryl amide and N-methylol(meth)acryl amide; vinyl esters, such as vinyl acetate and vinyl propionate; vinyl ethers, such as methyl vinyl ether; α-olefins, such as ethylene and propylene; halogenated α,β-unsaturated aliphatic monomers, such as vinyl chloride and vinylidene chloride; α,β-unsaturated aromatic monomer, such as styrene and α-methylstyrene; and the like. These substances can be used singly or in combination of two or more kinds thereof.

The polyisocyanate includes, for example, aliphatic diisocyanates, such as tetramethylene diisocyanate, dodecamethylene diisocyanate, 1,4-butane diisocyanate, hexamethylene diisocyanate, 2,2,4-trimethylhexamethylene diisocyanate, 2,4,4-trimethylhexamethylene diisocyanate, lysine diisocyanate, 2-methylepentane-1,5-diisocyanate, and 3-methylepentane-1,5-diisocyanate; alicyclic diisocyanates, such as isophorone diisocyanate, hydrogenated xylylene diisocyanate, 4,4′-cyclohexylmethane diisocyanate, 1,4-cyclohexane diisocyanate, methylcyclohexylene diisocyanate, and 1,3-bis(isocyanatomethyl)cyclohexane; aromatic diisocyanates, such as tolylene diisocyanate, 2,2′-diphenylmethane diisocyanate, 2,4′-diphenylmethane diisocyanate, 4,4′-diphenylmethane diisocyanate, 4,4′-diphenyl dimethylmethane diisocyanate, 4,4′-dibenzyl diisocyanate, 1,5-naphthylene diisocyanate, xylylene diisocyanate, 1,3-phenylene diisocyanate, and 1,4-phenylene diisocyanate; aromatic aliphatic diisocyanates, such as dialkyl diphenylmethane diisocyanate, tetraalkyl diphenylmethane diisocyanate, and α,α,α,α-tetramethylxylylene diisocyanate, and the like. These substances can be used singly or in combination of two or more kinds thereof.

The polyisocyanate can also be prepared as a denatured substance insofar as the effects of the present invention are not impaired. The polyisocyanate denatured substance includes, for example, multimers (dimers (for example, a uretdione denatured substance and the like), trimmers (for example, an isocyanurate denatured substance, an iminooxadiazinedione denatured substance, and the like) and the like), buret denatured substances (for example, a buret denatured substance generated by a reaction with water and the like), allophanate denatured substances (for example, allophanate denatured substances generated by a reaction with a mono-ol or low molecular weight polyol and the like), polyol denatured substances (for example, polyol denatured substance generated by a reaction with low molecular weight polyol or high molecular weight polyol and the like), oxadiazinetrion denatured substances (for example, oxadiazinetrion generated by a reaction with carbon dioxide), carbodiimide denatured substances (carbodiimide denatured substance generated by a decarbonization acid condensation reaction and the like), and the like but the invention not limited thereto.

Moreover, to the polyols or the polyisocyanates, a proper amount of a catalyst which promotes a reaction of a hydroxyl group of the polyol and an isocyanate group of the polyisocyanate may be added. As the catalyst, a known urethanization catalyst can be used. As a specific example of the catalyst, organometallic compounds, such as dibutyltin dilaurate, dibutyltin diacetate, and dioctyltin dilaurate, organic amines, such as triethylene diamine and triethyl amine, and salts thereof are selected and used. These catalysts can be used singly or in combination of two or more kinds thereof.

### Other Additives

To the photoacoustic blood model of the present invention, a scatterer and a plasticizer may be added as other additives as appropriate, as required.

The scatterer is a compound having light scattering properties and is added for approximation to the light propagation characteristics of human tissues and can adjust the equivalent scattering coefficient.

As the compound having light scattering properties, inorganic particles can be suitably used. As the inorganic particles, inorganic particles having small absorption in the used wavelength band of a photoacoustic wave diagnosing apparatus can be selected as appropriate. In order to scatter light, the refractive index is desirably different from that of the blood model base material. In order to achieve scattering of the inorganic particles, the average particle diameter is suitably 100 nm or more and more suitably 200 nm or more. Such inorganic particles suitably contain silicon oxide, metal oxide, a composite metal oxide, a metallic compound semiconductor, metal, or diamond. Examples of the metal oxide include aluminum oxide, titanium oxide, niobium oxide, tantalum oxide, zirconium dioxide, zinc oxide, magnesium oxide, tellurium oxide, yttrium oxide, indium oxide, tin oxide, indium oxide tin, and the like. Examples of the composite metal oxide include lithium niobate, potassium niobate, lithium tantalate, and the like. Examples of the metallic compound semiconductor include metal sulfides, such as zinc sulfide and cadmium sulfide, zinc selenide, cadmium selenide, zinc telluride, cadmium telluride, and the like. Examples of the metal include gold and the like.

The inorganic particles may be surface-treated. For example, so-called core-shell type inorganic particles in which one kind of inorganic particles are covered with another inorganic component can also be used. Since the titanium oxide has activity induced by light, the titanium oxide is suitably subjected to modification treatment of covering the surface with inorganic components, such as silica and alumina. Moreover, in order to improve the dispersibility into the blood model base material which is an organic substance, a dispersion assistant having an organic component may be used. The dispersion assistant having an organic component is not particularly limited insofar as it has compatibility with the blood model base material. The shape of the inorganic particles may be any shape of a spherical shape, an oval shape a flat shape, and a rod shape.

As the plasticizer, known plasticizers can be used for the purpose of adjusting the viscosity as a fluid. The known plasticizers include phthalate, trimellitate, pyromellitate, aliphatic monobasic acid ester, aliphatic dibasic acid ester, phosphate, esters of polyhydric alcohols, and the like. These substances can be used singly or in combination of two or more kinds thereof.

The phthalate includes dimethyl phthalate, diethyl phthalate, dipropyl phthalate, diisopropyl phthalate, dibutyl phthalate, diisobutyl phthalate, diamyl phthalate, di-n-hexyl phthalate, dicyclohexyl phthalate, diheptyl phthalate, di-n-octyl phthalate, dinonyl phthalate, diisononyl phthalate, diisodecyl phthalate, diundecyl phthalate, ditridecyl phthalate, diphenyl phthalate, di(2-ethylhexyl)phthalate, di(2-butoxyethyl)phthalate, 2-ethylhexyl benzyl phthalate, n-butyl benzyl phthalate, isononoyl benzyl phthalate, dimethyl isophthalate, and the like.

The trimellitate includes tributyl trimellitate, trihexyl trimellitate, tri-n-octyl trimellitate, tri-2-ethylhexyl trimellitate, triisodecyl trimellitate, and the like.

The pyromellitate includes tetrabutyl pyromellitate, tetrahexyl pyromellitate, tetra-n-octyl pyromellitate, tetra-2-ethylhexyl pyromellitate, tetradecyl pyromellitate, and the like.

The aliphatic monobasic acid ester includes butyl oleate, methyl oleate, methyl octanoate, butyl octanoate, methyl dodecanoate, butyl dodecanoate, methyl palmitate, butyl pulmitate, methyl stearate, butyl stearate, methyl linolate, butyl linolate, methyl isostearate, butyl isostearate, methyl acetyl ricinolate, butyl acetyl ricinolate, and the like.

The aliphatic dibasic acid ester includes dimethyl adipate, diethyl adipate, di-n-propyl adipate, diisopropyl adipate, diisobutyl adipate, di-n-octyl adipate, di-(2-ethylhexyl)adipate, diisononyl adipate, diisodecyl adipate, di(2-butoxyethyl)adipate, di(butyldiglycol)adipate, hepthylnonyl adipate, dimethyl azelate, di-n-octyl azelate, di(2-ethylhexyl)azelate, diethyl succinate, dimethyl sebacate, diethyl sebacate, dibutyl sebacate, di-n-octyl sebacate, di(2-ethylhexyl)sebacate, dibutyl phmalate, di(2-ethylhexyl)phmalate, dimethyl maleate, diethyl maleate, di-n-butyl maleate, di(2-ethylhexyl)maleate, and the like.

The phosphate ester includes trimethyl phosphate, triethyl phosphate, tributyl phosphate, tri-n-amyl phosphate, triphenyl phosphate, tri-o-cresyl phosphate, trixylenyl phosphate, 2-ethylhexyl diphenyl phosphate, cresyl diphenyl phosphate, tris(2-butoxyethyl)phosphate, tris(2-ethylhexyl)phosphate, and the like.

The esters of polyhydric alcohols include diethylene glycol diacetyrate, diethylene glycol dibenzoate, glycerol mono-oleate, glycerol tributyrate, glycerol triatetate, glyceryl-tri(acetylricinolate), triethylene glycol diacetate, and the like.

### Phantom for Photoacoustic Wave Diagnosing Apparatus

By disposing the photoacoustic blood model of the present invention in a phantom for a photoacoustic wave diagnosing apparatus, accuracy control and calibration of the diagnosing apparatus can be performed.

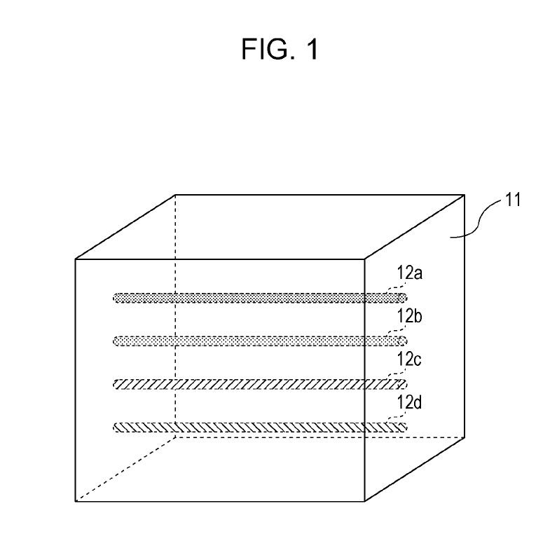

FIG. 1 is a view illustrating a configuration example of a phantom for a photoacoustic wave diagnosing apparatus using the photoacoustic blood model of the present invention. Blood models 12a to 12d serving as simulated tumors are disposed in a phantom base material 11. The size of the phantom is 120×120×50 mm. The size of the blood models 12a to 12d disposed in the phantom is 2 mm in diameter and 120 mm in length and are disposed in such a manner as to be able to be detected at a depth position of 25 mm when disposing the apparatus.

The phantom base material 11 is desirably a material whose acoustic propagation characteristics are similar to those of a living body and whose acoustic velocity is 800 m/s or more and 2000 m/s or less. For example, a material having an acoustic velocity of 1300 m/s or more and 1700 m/s or less, such as polyurethane gel and natural rubber, is particularly desirable. As the polyurethane gel, the same substances as those mentioned as the polyurethane gel for use in the blood model base material are mentioned. Moreover, in order to adjust the light scattering characteristics and the acoustic characteristics, inorganic particles and a plasticizer can be added as additives to the polyurethane gel.

In the blood model of the present invention, the parameter S calculated and determined from Expression (1) can be controlled between 0 to 100. By installing the phantom in a photoacoustic wave diagnosing apparatus, and performing measurement, it can be confirmed that the diagnosing apparatus measures the correct oxygen saturation and also it is possible to perform calibration of the apparatus based on the measurement value.

### Photoacoustic Fluid Blood Model

The photoacoustic blood model of the present invention may be a photoacoustic fluid blood model (hereinafter sometimes referred to as a “fluid blood model”) which is liquid having fluidity.

### Light Absorbing Compound

The light absorbing compound is as described in the section of “Photoacoustic blood model” above.

### Fluid Blood Model Base Material

In the photoacoustic fluid blood model, when the fluid blood models are irradiated with light having a certain wavelength λ, the fluid blood models thermally expand according to the absorption coefficient, so that acoustic waves (generally ultrasonic waves) are generated. Between the intensity P of the acoustic wave to be obtained, the intensity F of the laser light in that case, and the absorption coefficient μ, the relationship of P=Γ·μ·F is established. Γ is referred to as a Gruneisen coefficient and is a constant peculiar to materials.

In the present invention, the absorption coefficient of the light absorber is required to be adjusted by compounding the light absorbing compound in the fluid blood model base material. Therefore, as the fluid blood model base material simple substance, in the used wavelength band of the photoacoustic wave diagnosing apparatus, the light absorption is suitably small and transparent.

Since the acoustic velocity of biological tissues is in the range of about 1000 m/s to 1700 m/s, the acoustic velocity of the fluid blood model base material is suitably 800 m/s or more and 2000 m/s or less and more suitably 1300 m/s or more and 1700 m/s or less particularly from the similarity of the acoustic propagation to soft tissues.

Moreover, the Gruneisen coefficient Γ has a relationship of Γ=β·v²/Cp (β: Coefficient of thermal volume expansion, v: Acoustic velocity, Cp: Specific heat at constant pressure). The coefficient of thermal volume expansion β and the specific heat at constant pressure Cp of the fluid blood model base material is suitably in the range where the Gruneisen coefficient Γ does not deviate from that of a living body

For the fluid blood model of the present invention, organic solvents, such as water and alcohol, can be used as the fluid blood model base material in order to express fluidity similarly as blood. From the viewpoint of the stability of the fluid blood model, the fluid blood model base material is suitably nonvolatile and particularly polyol is particularly suitable because the acoustic velocity is 1500 m/s and is also similar to that of a living body.

The polyol includes polyester polyol, polyether polyol, and the like, for example. These substances can be used singly or in combination of two or more kinds thereof. As the polyester polyol and the polyether polyol, the same substances mentioned as the polyester polyol and the polyether polyol for use in the blood model base material are mentioned.

### Other Additives

Other additives are as described in the section of “Photoacoustic blood model” above.

### Phantom for Photoacoustic Wave Diagnosing Apparatus Employing Fluid Blood Model

By disposing the photoacoustic blood model of the present invention in a phantom for a photoacoustic wave diagnosing apparatus, accuracy control and calibration of the diagnosing apparatus can be performed.

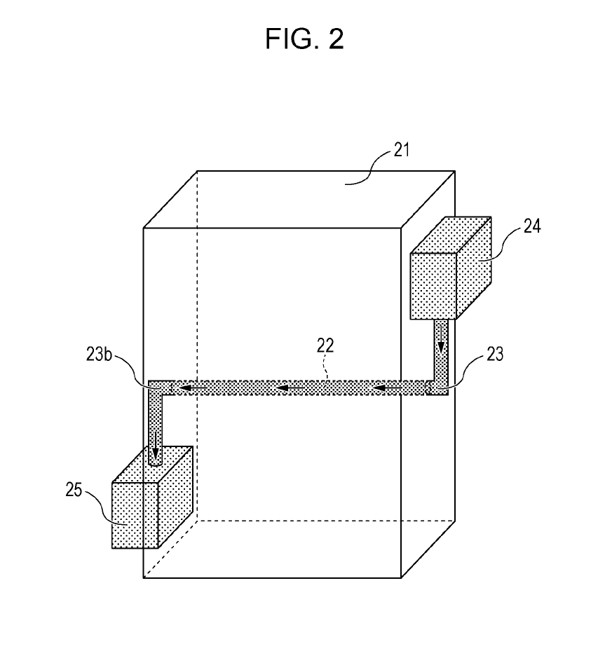

FIG. 2 illustrates a configuration example of a phantom for a photoacoustic wave diagnosing apparatus employing the photoacoustic fluid blood model of the present invention. A hole 22 which simulates a blood vessel is disposed in a phantom base material 21, and the fluid blood model of the present invention is passed thereinto. 23a and 23b each denotes pipes for sending the fluid blood model and are connected to a pump 24 and a waste liquid reservoir 25, respectively. The size of the phantom is 120×120×50 mm. The size of the hole 22 which simulates a blood vessel to be disposed in the phantom is 2 mm in diameter and 120 mm in length and is disposed in such a manner as to be able to be detected at a depth position of 25 mm when disposing the apparatus. In FIG. 2, although the straight line-like hole is arranged, the hole may simulate an actual blood vessel and have a curved shape. Moreover, the size of the phantom can also be adjusted as appropriate according to the size of the apparatus. In FIG. 2, the pump 24 for liquid sending and the waste liquid reservoir 25 are illustrated but the pipe 23b for sending the fluid blood model can be connected to the pump 24 to form a circular system.

The phantom base material 21 is desirably a material whose acoustic propagation characteristics are similar to those of a living body and whose acoustic velocity is 800 m/s or more and 2000 m/s or less. For example, a material having an acoustic velocity of 1300 m/s or more and 1700 m/s or less, such as polyurethane gel and natural rubber, is particularly desirable. As the polyurethane gel, the same substances as those mentioned as the polyurethane gel for use in the blood model base material are mentioned. Moreover, in order to adjust the light scattering characteristics and the acoustic characteristics, inorganic particles and a plasticizer can be added as an additive to a polyurethane gel.

In the fluid blood model of the present invention, the parameter S calculated and determined from Expression (1) can be controlled between 0 to 100. By installing the phantom in a photoacoustic wave diagnosing apparatus, and performing measurement, it can be confirmed that the diagnosing apparatus measures the correct oxygen saturation and the flow velocity and also it is possible to perform calibration of the apparatus based on the measurement values.

### EXAMPLES

Hereinafter, examples are described in order to describe the present invention in detail but the present invention is not limited to these examples.

### Examples 1 to 4, Comparative Examples 1 and 2

### Preparation of Blood Model Test Piece

Two kinds of light absorbing compounds and a compound having light scattering properties were dispersed in a beaker in which polyol was placed, stirred, and then subjected to vacuum defoaming.

As the polyol, a polyether polyol copolymer (number average molecular weight of 6000) having a molar ratio of ethylene oxide and propylene oxide of 1:1 was used.

As the light absorbing compound, carbon black and copper phthalocyanine (maximum absorption wavelength of 721 nm) were used. With respect to the carbon black, a paste (carbon black content of 25% by weight) in which carbon black was dispersed in the same polyol as the polyol in the beaker was added in such a manner that the paste content in a blood model was as shown in Table 1. With respect to the copper phthalocyanine, a paste (copper phthalocyanine content of 20% by weight) in which copper phthalocyanine was dispersed in the same polyol as the polyol in the beaker was added in such a manner that the paste content in a blood model was as shown in Table 1.

As the compound having light scattering properties, titanium oxide (average particle diameter of 0.21 μm) which was surface-treated with aluminum oxide and hexamethyldisilazane was dispersed in a proportion of 0.2% by weight based on polyol.

Next, hexamethylene diisocyanate-modified polyisosicanate serving as a curing agent was added in a proportion of 3.4% by weight based on polyol, stirred, and then subjected to vacuum defoaming. A polyurethane gel mixed solution thus prepared was poured into a mold, and then heated at 90° C. for 1 hour to be cured. Thereafter, the cured substance was released from the mold to thereby obtain a blood model test piece for use in each measurement described below.

### Calculation of Absorption Coefficient of Blood Model

In a 50 mm×50 mm quartz cell having an optical path length of 5 mm, the blood model was cured to thereby prepare a cell for measuring the absorption coefficient. The transmittance and the reflectance of the cell were determined using a spectrum photometer (manufactured by Jasco Corp., V-670). Separately, the refractive index of the blood model test piece of 10×10×50 mm was determined using a refractive index meter (manufactured by Shimadzu, KPR-2000). With respect to these results, optimization of variable setting was performed by Monte Carlo simulation in such a manner that a difference between a measured value and a calculated value was the minimum, and then the absorption coefficient at each wavelength (λ₁=756 nm, λ₂=799 nm) were calculated. The results are shown in Table 1.

### Calculation of Parameter S′

A parameter S′ was determined from Expression (1′) using the determined absorption coefficient of the blood model. The results are shown in Table 1.

### Measurement of Photoacoustic Signal Intensity

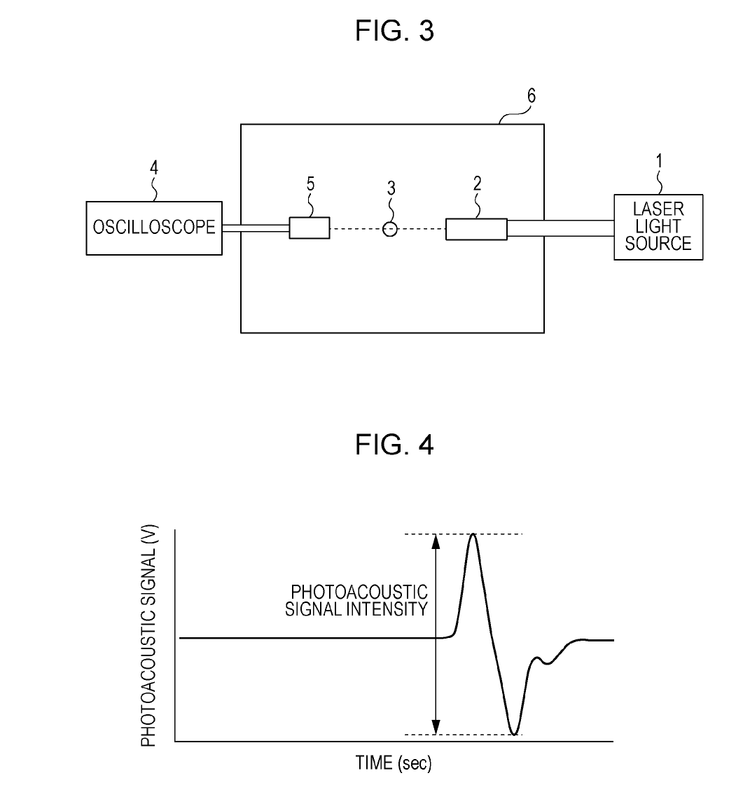

FIG. 3 is an outline view of a photoacoustic signal intensity measuring device.

A test piece 3 was irradiated with laser through an optical fiber 2 using titanium sapphire laser (manufactured by Lotis Tii, LT-2211) as a laser light source 1 under the conditions of wavelengths of 756 nm and 797 nm, an energy density of 20 mJ/cm², a pulse width of 20 nanosecond, and a pulse repetition of 10 Hz. As the test piece 3, a tube-like blood model test piece 2 mm in diameter and 200 mm in length was used. The test piece 3 was placed in a water tank 6 without causing bending in the test piece 3. Acoustic waves generated by irradiating the test piece 3 with laser light were received by an ultrasonic transducer (manufactured by Olympus NDT Inc., V303 (center frequency of 1 MHz)) which is a receiving device 5. The received voltage value of a photoacoustic signal received by the receiving device 5 was measured using an oscilloscope 4 (manufactured by LeCroy Japan, WaveRunner 64Xi).

The waveform of a typical photoacoustic signal is illustrated in FIG. 4. The obtained photoacoustic signal has a typical N-shaped waveform and the amplitude width of the maximum value and the minimum value is defined as the intensity of the photoacoustic signal. The results are shown in Table 1.

### Calculation of Parameter S

The parameter S was calculated from Expression (1) using a ratio P′ (P₇₉₇/P₇₅₆) of the obtained photoacoustic signal intensities P₇₅₆ and P₇₉₇. The results are shown in Table 1.

### Calculation of Acoustic Velocity

An ultrasonic wave transducer (transmitting portion) as a probe used in the measurement of the photoacoustic signal intensity and a hydrophone (receiving portion) (manufactured by Toray Engineering Co., Ltd., Needle type hydrophone) were used. The transducer and the hydrophone were fixed with a jig in the water tank in such a manner that the center of the acoustic axis of the transducer and the center of the acoustic axis of the hydrophone were in agreement with each other. The distance between the transducer and the hydrophone was 40 mm.

As the blood model test piece, a plate-like test piece of 100 mm×100 mm×10 mm was used. The test piece was fixed between the transducer and the hydrophone using a jig in such a manner that the incidence angle of an ultrasonic wave signal to the test piece was 0°. A sign wave (Transmission voltage of 100 V) of 8 cycles was transmitted from the transducer using a function generator (manufactured by NF Corporation, WF1946), and then the received voltage value of the hydrophone when disposing each test piece was determined using an oscilloscope (manufactured by LeCroy Japan, WaveRunner 64Xi). A difference in the received wave arrival time between a case where the test piece was placed in the measurement system and a case where the test piece was not placed in the measurement system was determined by taking the crossing correlation of the waveforms obtained by the oscilloscope, and then the acoustic velocity was determined from the difference in the received wave arrival time. The results are shown in Table 1.

### Measurement of Coefficient of Linear Thermal Expansion

The coefficient of linear thermal expansion was measured based on the coefficient of linear thermal expansion test method (JIS-K7197) by thermomechanical analysis of plastic. Specifically, as a blood model test piece, a cylindrical test piece 4 mm in diameter and 5 mm in height was used. The test piece was placed in a thermomechanical analyzer (manufactured by Rigaku Corporation, Thermo Plus EVO TMA8310), and then temperature raising and temperature lowering of −40° C. to 60° C. were repeated twice under the conditions of a temperature rise rate of 5° C. under a nitrogen flow (100 mL/min). The average coefficient of linear thermal expansion in the temperature range of 0 to 25° C. at the second temperature raising was calculated. The results are shown in Table 1.

TABLE 1

| | | Copper | | | | | | | | Coefficient of |
|---|---|---|---|---|---|---|---|---|---|---|
| | Carbon black | phthalocyanine | | | | | | | Acoustic | linear thermal |
| | paste content | paste content | μa[756] | μa[797] | | P₇₅₆ | P₇₉₇ | | velocity | expansion |
| | (% by weight) | (% by weight) | (mm⁻¹) | (mm⁻¹) | S′ | (V) | (V) | S | (m/s) | (ppm/k) |
| Ex. 1 | 0.004 | 0.013 | 0.0860 | 0.0566 | 35.38 | 0.5213 | 0.3550 | 39.47 | 1388 | 296.54 |
| Ex. 2 | 0.004 | 0.009 | 0.0743 | 0.0542 | 47.25 | 0.4512 | 0.3375 | 49.88 | 1386 | 293.35 |
| Ex. 3 | 0.005 | 0.006 | 0.0799 | 0.0657 | 59.52 | 0.5012 | 0.3991 | 56.30 | 1391 | 291.15 |
| Ex. 4 | 0.005 | 0.003 | 0.0774 | 0.0710 | 69.41 | 0.4575 | 0.4143 | 68.30 | 1390 | 297.24 |
| Comp. | 0.010 | 0.000 | 0.1351 | 0.1315 | 74.41 | 1.3190 | 1.2188 | 73.25 | 1388 | 295.98 |
| Ex. 1 | | | | | | | | | | |
| Comp. | 0.002 | 0.000 | 0.0322 | 0.0309 | 73.24 | 0.4080 | 0.3917 | 70.02 | 1387 | 291.94 |
| Ex. 2 | | | | | | | | | | |

As shown in Table 1, it is found that the blood model prepared only by carbon black cannot control the parameter S but the parameter S can be controlled by the use of carbon black and copper phthalocyanine. More specifically, it was found that the photoacoustic blood model of the present invention can control the parameter S and it was clarified that the photoacoustic blood model of the present invention can be used for accuracy control and calibration of a photoacoustic wave diagnosing apparatus. Moreover, S′ and S were well agreement with each other in both Examples and Comparative Examples.

### Examples 5 to 7

34.570 g of polytetramethylene ether glycol (PTMG, number average molecular weight of 2000) and 8.650 g of a plasticizer (diisononyl phthalate, DINP) were mixed to prepare a polyol solution.

The following phthalocyanine compound was dissolved in the polyol solution with the amount shown in Table 2 using mechanical stirring with supersonic treatment and a propeller.

Copper phthalocyanine (FD-25c, maximum absorption wavelength of 829 nm, manufactured by Yamada Chemical Co., Ltd.) Phthalocyanine vanadium complex (FD-43, maximum absorption wavelength of 754 nm, manufactured by Yamada Chemical Co., Ltd.)

With the phthalocyanine compound containing polyol solution, 0.005 g of a urethanization catalyst (dibutyltin dilaurate) and 6.775 g of a curing agent (hexamethylene diisocyanate trimer) were sufficiently mixed, poured into a mold, and then heated at 90° C. for 2 hours. Thereafter, the resultant substance was released from the mold to obtain a blood model test piece. The results of evaluating the test piece in the same manner as in Examples 1 to 4 are shown in Table 2.

TABLE 2

| | Copper | Phthalocyanine | | | | | | | | Coefficient of |
|---|---|---|---|---|---|---|---|---|---|---|
| | phthalocyanine | vanadium | | | | | | | Acoustic | linear thermal |
| | paste content | complex content | μa [756] | μa [797] | | P₇₅₆ | P₇₉₇ | | velocity | expansion |
| | (% by weight) | (% by weight) | (mm⁻¹) | (mm⁻¹) | S′ | (V) | (V) | S | (m/s) | (ppm/k) |
| Ex. 5 | 0.00026258 | 0.000011631 | 0.0188 | 0.0254 | 97.3 | 0.1256 | 0.1512 | 90.0 | 1496 | 242.16 |
| Ex. 6 | 0.00026135 | 0.000024588 | 0.0257 | 0.0255 | 75.9 | 0.1747 | 0.1747 | 76.6 | 1496 | 257.34 |
| Ex. 7 | 0.00026013 | 0.000037546 | 0.0327 | 0.0256 | 54.8 | 0.2010 | 0.1718 | 63.1 | 1498 | 244.17 |

The results of Table 2 showed that the photoacoustic blood models in which S′ and S were well in agreement with each other and the S value was 0 or more 100 or less were obtained by the use of the light absorbing compound whose maximum absorption wavelength was λ₁ (756 nm) or less and the light absorbing compound whose maximum absorption wavelength was λ₂ (797 nm) or more. Therefore, it was clarified that the photoacoustic blood model of the present invention can be used for accuracy control and calibration of a photoacoustic diagnosing apparatus.

With respect to the copper phthalocyanine used in this example, μ[λ₂]/μ[λ₁] was 2.0 and, in the case of a simple substance, the parameter S was 117. With respect to the phthalocyanine vanadium complex used in this example, μ[λ₂]/μ[λ₁] was 0.04 and, in the case of a simple substance, the parameter S was −1780. Therefore, it was found that, by changing the content ratio of the two kinds of phthalocyanine compounds, the value of the parameter S can be freely controlled to be 0 or more and 100 or less.

### Examples 8 to 14, Comparative Examples 3 and 4

### Preparation of Fluid Blood Model

Two kinds of light absorbing compounds and a compound having light scattering properties were dispersed in a beaker in which polyol as the fluid blood model base material was placed, stirred, and then subjected to vacuum defoaming to prepare a fluid blood model.

As the polyol, a polyether polyol copolymer (number average molecular weight of 5000) having a molar ratio of ethylene oxide and propylene oxide of 1:1 was used.

As the light absorbing compound, carbon black and copper phthalocyanine were used. With respect to the carbon black, a paste (carbon black content of 25% by weight) in which carbon black was dispersed in the same polyol as the polyol in the fluid blood model base material was added in such a manner that the paste content in a fluid blood model was as shown in Table 3. With respect to the copper phthalocyanine, a paste (copper phthalocyanine content of 20% by weight) in which copper phthalocyanine was dispersed in the same polyol as the polyol in the fluid blood model base material was added in such a manner that the paste content in a fluid blood model was as shown in Table 3.

As the compound having light scattering properties, titanium oxide (average particle diameter of 0.21 μm) which was surface-treated with aluminum oxide and hexamethyldisilazane was dispersed in a proportion of 0.2% by weight based on polyol.

### Calculation of Absorption Coefficient of Fluid Blood Model

Into a 50 mm×50 mm quartz cell having an optical path length of 5 mm, the fluid blood model to which a curing agent was added was poured, and then heated at 90° C. for 1 hour to cure the resin to thereby prepare a cell for measuring the absorption coefficient. The transmittance and the reflectance of the cell were determined using a spectrum photometer (manufactured by Jasco Corp., V-670). Separately, the refractive index of a sample (size of 10×10×50 mm) which was similarly subjected to resin curing was determined using a refractive index meter (manufactured by Shimadzu, KPR-2000). With respect to these results, optimization of variable setting was performed by Monte Carlo simulation in such a manner that a difference between a measured value and a calculated value was the minimum, and then the absorption coefficient at each wavelength (λ₁=756 nm, λ₂=799 nm) was calculated. The results are shown in Table 3.

### Calculation of Parameter S

A parameter S′ was determined from Expression (1′) using the determined absorption coefficient of the fluid blood model to be used as the parameter S. The results are shown in Table 3.

TABLE 3

| | | Copper | | | |
|---|---|---|---|---|---|
| | Carbon black | phthalocyanine | | | |
| | paste content | paste content | μa[756] | μa[797] | |
| | (% by weight) | (% by weight) | (mm⁻¹) | (mm⁻¹) | S |
| Ex. 8 | 0.010 | 0.055 | 0.2689 | 0.1498 | 13.74 |
| Ex. 9 | 0.012 | 0.040 | 0.2662 | 0.1738 | 34.46 |
| Ex. 10 | 0.004 | 0.014 | 0.0904 | 0.0604 | 37.32 |
| Ex. 11 | 0.004 | 0.009 | 0.0773 | 0.0569 | 48.19 |
| Ex. 12 | 0.005 | 0.007 | 0.0815 | 0.0656 | 57.41 |
| Ex. 13 | 0.020 | 0.027 | 0.3623 | 0.3125 | 63.96 |
| Ex. 14 | 0.005 | 0.003 | 0.0810 | 0.0741 | 69.19 |
| Comp. Ex. 3 | 0.010 | 0.000 | 0.1345 | 0.1342 | 76.40 |
| Comp. Ex. 4 | 0.002 | 0.000 | 0.0337 | 0.0329 | 74.65 |

As shown in Table 3, it is found that the blood model prepared only by carbon black cannot control the parameter S but the parameter S can be controlled by the use of carbon black and copper phthalocyanine. More specifically, it was found that the photoacoustic fluid blood model of the present invention can control the parameter S and it was clarified that the photoacoustic fluid blood model of the present invention can be used for accuracy control and calibration of a photoacoustic wave diagnosing apparatus.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application Nos. 2013-108702, filed May 23, 2013, 2013-108703, filed May 23, 2013, and 2014-044541 filed Mar. 7, 2014, which are hereby incorporated by reference herein in their entirety.

1. A photoacoustic blood model, comprising:
two or more kinds of light absorbing compounds in a blood model base material,
wherein absorption coefficient ratios μ[λ₂]/μ[λ₁] at arbitrary two wavelengths λ₁ and λ₂ (λ₁<λ₂) of 600 nm or more and 1100 nm or less of the light absorbing compounds are different from each other and a parameter S calculated from the following equation (1) is 0 or more and 100 or less, and
wherein a coefficient of linear thermal expansion of the blood model base material is 100 ppm/K or more and 1000 ppm/K or less,
$$\begin{matrix}[ Math . 1 ] & \\ S = \frac{{P}^{′} \cdot Hb [ {λ}_{1} ] - Hb [ {λ}_{2} ]}{\begin{matrix}( {HbO}_{2} [ {λ}_{2} ] - Hb [ {λ}_{2} ] ) - \\ {P}^{′} \cdot ( {HbO}_{2} [ {λ}_{1} ] - Hb [ {λ}_{1} ] )\end{matrix}} \cdot 100 & Expression ( 1 )\end{matrix}$$
wherein HbO₂[λ₁] indicates an absorption coefficient of oxyhemoglobin at the wavelength λ₁, HbO₂[λ₂] indicates an absorption coefficient of oxyhemoglobin at the wavelength λ₂, Hb[λ₁] indicates an absorption coefficient of deoxyhemoglobin at the wavelength λ₁, Hb[λ₂] indicates an absorption coefficient of deoxyhemoglobin at the wavelength λ₂, and P′ indicates a ratio (P_{λ2}/P_{λ1}) of a photoacoustic signal intensity P_{λ2} obtained by irradiation with light of the wavelength λ₂ to a photoacoustic signal intensity P_{λ1} obtained by irradiation with light of the wavelength λ₁.

2. The photoacoustic blood model according to claim 1, comprising at least one light absorbing compound in which the absorption coefficient ratio μ[λ₂]/μ[λ₁] satisfies one of the following expression (2) or expression (3),
$$\begin{matrix}[ Math . 2 ] & \\ {S}_{min} \geq \frac{( μ [ {λ}_{2} ] / μ [ {λ}_{1} ] ) \cdot Hb [ {λ}_{1} ] - Hb [ {λ}_{2} ]}{\begin{matrix}( {HbO}_{2} [ {λ}_{2} ] - Hb [ {λ}_{2} ] ) - \\ ( μ [ {λ}_{2} ] / μ [ {λ}_{1} ] ) \cdot ( {HbO}_{2} [ {λ}_{1} ] - Hb [ {λ}_{1} ] )\end{matrix}} \cdot 100 & Expression ( 2 ) \\ {S}_{max} \leq \frac{( μ [ {λ}_{2} ] / μ [ {λ}_{1} ] ) \cdot Hb [ {λ}_{1} ] - Hb [ {λ}_{2} ]}{\begin{matrix}( {HbO}_{2} [ {λ}_{2} ] - Hb [ {λ}_{2} ] ) - \\ ( μ [ {λ}_{2} ] / μ [ {λ}_{1} ] ) \cdot ( {HbO}_{2} [ {λ}_{1} ] - Hb [ {λ}_{1} ] )\end{matrix}} \cdot 100 & Expression ( 3 )\end{matrix}$$
wherein Sₘᵢₙ indicates a lower limit of the parameter S, Sₘₐₓ indicates an upper limit of the parameter S, HbO₂[λ₁] indicates an absorption coefficient of oxyhemoglobin at the wavelength λ₁, HbO₂[λ₂] indicates an absorption coefficient of oxyhemoglobin at the wavelength λ₂, Hb[λ₁] indicates an absorption coefficient of deoxyhemoglobin at the wavelength λ₁, Hb[λ₂] indicates an absorption coefficient of deoxyhemoglobin at the wavelength λ₂, μ[λ₁] indicates an absorption coefficient of the light absorbing compound at the wavelength λ₁, and μ[λ₂] indicates an absorption coefficient of the light absorbing compound at the wavelength λ₂.

3. The photoacoustic blood model according to claim 2, further comprising at least one light absorbing compound in which the absorption coefficient ratio μ[λ₂]/μ[λ₁] satisfies the other one of the expression (2) or the expression (3).

4. (canceled)

5. The photoacoustic blood model according to claim 1, wherein an acoustic velocity of the blood model base material is 800 m/s or more and 2000 m/s or less.

6. The photoacoustic blood model according to claim 5, wherein the acoustic velocity of the blood model base material is 1300 m/s or more and 1700 m/s or less.

7. The photoacoustic blood model according to claim 1, wherein the blood model base material contains a polymer material.

8. The photoacoustic blood model according to claim 7, wherein the blood model base material is a polyurethane gel.

9. A photoacoustic blood model, comprising:
two or more kinds of light absorbing compounds in a blood model base material,
wherein absorption coefficient ratios μ[λ₂]/μ[λ₁] at arbitrary two wavelengths λ₁ and λ₂(λ₁<λ₂) of 600 nm or more and 1100 nm or less of the light absorbing compounds are different from each other and a parameter S calculated from the following equation (1) is 0 or more and 100 or less,
wherein a coefficient of linear thermal expansion of the blood model base material is 100 ppm/K or more and 1000 ppm/K or less, and
wherein the blood model base material is nonvolatile,
$$\begin{matrix}[ Math . 1 ] & \\ S = \frac{{P}^{′} \cdot Hb [ {λ}_{1} ] - Hb [ {λ}_{2} ]}{\begin{matrix}( {HbO}_{2} [ {λ}_{2} ] - Hb [ {λ}_{2} ] ) - \\ {P}^{′} \cdot ( {HbO}_{2} [ {λ}_{1} ] - Hb [ {λ}_{1} ] )\end{matrix}} \cdot 100 & Expression ( 1 )\end{matrix}$$
wherein HbO₂[λ₁] indicates an absorption coefficient of oxyhemoglobin at the wavelength λ₁, HbO₂[λ₂] indicates an absorption coefficient of oxyhemoglobin at the wavelength λ₂, Hb[λ₁] indicates an absorption coefficient of deoxyhemoglobin at the wavelength λ₁, Hb[λ₂] indicates an absorption coefficient of deoxyhemoglobin at the wavelength λ₂, and P′ indicates a ratio (P_{λ2}/P_{λ1}) of a photoacoustic signal intensity P_{λ2} obtained by irradiation with light of the wavelength λ₂ to a photoacoustic signal intensity P_{λ1} obtained by irradiation with light of the wavelength λ₁.

10. The photoacoustic blood model according to claim 9, wherein the blood model base material is polyol.

11. The photoacoustic blood model according to claim 1, wherein at least one of the light absorbing compounds is a phthalocyanine compound.

12. The photoacoustic blood model according to claim 11, wherein at least two of the light absorbing compounds are phthalocyanine compounds.

13. The photoacoustic blood model according to claim 11, wherein the phthalocyanine compound is selected from copper phthalocyanine and a phthalocyanine vanadium complex.

14. The photoacoustic blood model according to claim 11, wherein each of the phthalocyanine compounds is contained in a proportion of 0.0000001% by weight or more and 0.1% by weight or less.

15. The photoacoustic blood model according to claim 1, wherein at least one of the light absorbing compounds is carbon black.

16. The photoacoustic blood model according to claim 1, wherein the arbitrary two wavelengths λ₁ and λ₂ are λ₁=756 nm and λ₂=799 nm.

17. A phantom for a photoacoustic wave diagnosing apparatus, comprising:
a photoacoustic blood model; and
a phantom base material,
said photoacoustic blood model, comprising:
two or more kinds of light absorbing compounds in a blood model base material,
wherein absorption coefficient ratios μ[λ₂]/μ[λ₁] at arbitrary two wavelengths λ₁ and λ₂(λ₁<λ₂) of 600 nm or more and 1100 nm or less of the light absorbing compounds are different from each other and a parameter S calculated from the following equation (1) is 0 or more and 100 or less, and
wherein a coefficient of linear thermal expansion of the blood model base material is 100 ppm/K or more and 1000 ppm/K or less,
$$\begin{matrix}[ Math . 1 ] & \\ S = \frac{{P}^{′} \cdot Hb [ {λ}_{1} ] - Hb [ {λ}_{2} ]}{\begin{matrix}( {HbO}_{2} [ {λ}_{2} ] - Hb [ {λ}_{2} ] ) - \\ {P}^{′} \cdot ( {HbO}_{2} [ {λ}_{1} ] - Hb [ {λ}_{1} ] )\end{matrix}} \cdot 100 & Expression ( 1 )\end{matrix}$$
wherein HbO₂[λ₁] indicates an absorption coefficient of oxyhemoglobin at the wavelength λ₁, HbO₂[λ₂] indicates an absorption coefficient of oxyhemoglobin at the wavelength λ₂, Hb[λ₁] indicates an absorption coefficient of deoxyhemoglobin at the wavelength λ₁, Hb[λ₂] indicates an absorption coefficient of deoxyhemoglobin at the wavelength λ₂, and P′ indicates a ratio (P_{λ2}/P_{λ1}) of a photoacoustic signal intensity P_{λ2} obtained by irradiation with light of the wavelength λ₂ to a photoacoustic signal intensity P_{λ1} obtained by irradiation with light of the wavelength λ₁.
